(19) United States
(12) Patent Application Publication
(10) Pub. No.: US 2024/0350403 A1
(43) Pub. Date: Oct. 24, 2024

(54) DEHYDRATED HERBIVORE THERAPEUTIC DELIVERY WAFER

(71) Applicant: Allen Bubela II, Utica, IL (US)

(72) Inventor: Allen Bubela II, Utica, IL (US)

(21) Appl. No.: 18/611,340

(22) Filed: Mar. 20, 2024

Related U.S. Application Data
(60) Provisional application No. 63/551,399, filed on Feb. 8, 2024.
(60) Provisional application No. 63/496,867, filed on Apr. 18, 2023.

(51) Int. Cl. A61K 9/00; A61K 31/4184; A61K 47/26; A61K 47/46

(52) U.S. Cl. CPC A61K 9/0056; A61K 31/4184; A61K 47/26; A61K 47/46

(57) ABSTRACT

Apparatus and associated methods relate to a dehydrated herbivore therapeutic delivery wafer. In an illustrative aspect, a dehydrated herbivore dewormer wafer may, for example, include an edible wafer composition including pieces of a first natural carbohydrate spread throughout the edible wafer configured such that the pieces of the first natural carbohydrate are visible on an exterior surface of the edible wafer. For example, the edible wafer may include a second natural carbohydrate coupled to at least some of the visible pieces of the first natural carbohydrate such that the natural carbohydrates may be sensed by an equine to be within the edible wafer composition. The edible wafer may include an active ingredient evenly distributed throughout the edible wafer composition. The edible wafer may include scorelines defining predetermined sections selectively separable from the edible composition wafer, configured such that each predetermined section comprises a predetermined amount of the active ingredient.

### CROSS-REFERENCE TO RELATED APPLICATIONS

This application is a nonprovisional application and claims the benefit of U.S. Application Ser. No. 63/496,867, titled “Dehydrated Wafer Equine Dewormer,” filed by Allen Bubela, et al., on Apr. 18, 2023.

This application is a nonprovisional application and claims the benefit of U.S. Application Ser. No. 63/551,399, titled “Dehydrated Wafer Equine Dewormer,” filed by Allen Bubela, et al., on Feb. 8, 2024.

This application incorporates the entire contents of the foregoing application herein by reference.

### TECHNICAL FIELD

Various embodiments generally relate to veterinary care.

### BACKGROUND

Horse treats, crucial for positive reinforcement and bonding, come in various formulations, including commercially available options with grains, fruits, and molasses, and homemade varieties with oats, carrots, and apples. Ingredients are chosen for nutritional balance and palatability, adhering to safety standards. These treats serve as effective positive reinforcement tools, leveraging the horse's intrinsic motivation for food rewards. Owners must exercise caution regarding size and consistency to mitigate choking hazards, ensuring a balanced nutritional intake.

Deworming horses is vital, involving anthelmintic medications every 6-8 weeks, tailored to individual needs. Fecal egg counts guide treatment for a targeted approach, and veterinarian collaboration is crucial for effective programs, accounting for age, health status, and environmental conditions. This proactive strategy prevents parasitic-related health issues while contributing to global efforts against resistance development in internal parasites.

Horses, as herbivores, thrive on a diet of plant-based materials, emphasizing continuous forage intake for digestive health. Their hindgut digestive system requires a high-fiber, low-calorie diet. In domestic settings, owners mimic natural behavior by providing quality forage, ensuring dental health, and monitoring individual nutritional needs. Recognizing horses as herbivores underscores the significance of a well-balanced diet aligned with evolutionary adaptations, promoting physical and mental health in captivity.

### SUMMARY

Apparatus and associated methods relate to a dehydrated herbivore therapeutic delivery wafer. In an illustrative aspect, a dehydrated herbivore dewormer wafer may, for example, include an edible wafer composition including pieces of a first natural carbohydrate spread throughout the edible wafer configured such that the pieces of the first natural carbohydrate are visible on an exterior surface of the edible wafer. The edible wafer may, for example, include a second natural carbohydrate coupled to at least some of the visible pieces of the first natural carbohydrate such that the natural carbohydrates may be sensed by an equine to be within the edible wafer composition. The edible wafer may, for example, include an active ingredient evenly distributed throughout the edible wafer composition. The edible wafer may, for example, include scorelines defining predetermined sections selectively separable from the edible composition wafer, configured such that each predetermined section comprises a predetermined amount of the active ingredient.

In some embodiments, the edible wafer may, for example, be configured wherein the herbivore is an equine. In some embodiments, the second natural carbohydrate may, for example, include a sweetener. The sweetener may, for example, include molasses. The sweetener may, for example, include an apple flavoring. The sweetener may, for example, include a carrot flavoring.

In some embodiments, the first natural carbohydrate may, for example, include grain. For example, the first natural carbohydrate and the second natural carbohydrate may, for example, physically bind together a base material, the active ingredient, and the pieces of the first natural carbohydrate. The base material may, for example, be configured to be at least 20% by weight of the edible wafer and include grain flour. For example, the active ingredient may, for example, include at 3% by weight of the edible wafer and include an anthelmintic. The anthelmintic may, for example, include fenbendazole.

In some embodiments, each section of the edible wafer includes a dosage of active ingredient for a predetermined weight of equine. The edible wafer may, for example, include a mold inhibitor spread throughout the wafer.

Various embodiments may achieve one or more advantages. For example, embodiments including pieces of carbohydrates and/or roughage (e.g., alfalfa, other fibrous materials known to appeal to a target herbivore) may advantageously appeal to the herbivore's natural diet. For example, embodiments including at natural carbohydrates in a manner they may be detected by an herbivore (taste, smell, texture, and/or sight) may appeal to the herbivore to consume the natural carbohydrates and the consume the edible including the active ingredient, acting as a medication.

The natural carbohydrates may, for example, be customized based on the herbivore's senses, such as quality of sight, quality of smell, quality of taste, quality of appreciation of food texture. For example, the natural carbohydrates within the wafer may, for example, be customized for a horse. For example, the natural carbohydrates within the wafer may, for example, be customized for an alpaca. The measure of the herbivore's sense of smell may, for example, be determined by factors such as the number of olfactory receptors for a sense of smell. For example, horses may have about 300 million olfactory receptors compared to humans with only about 5-6 olfactory receptors. The smell of the wafer may, for example, be an important decision point to a horse whether to consume the edible wafer.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary dehydrated wafer herbivore dewormer (DWHD) employed in an illustrative use-case scenario.

FIG. 2A depicts an exemplary dehydrated wafer herbivore dewormer batch contained within a tray.

FIG. 2B depicts an exemplary dehydrated wafer herbivore dewormer contained in a sealable bag.

FIG. 3A depicts an exemplary dehydrated wafer herbivore dewormer being weighed.

FIG. 3B depicts an exemplary dehydrated wafer herbivore dewormer score being weighed.

FIG. 3C depicts some different dehydrated wafer herbivore dewormer and dehydrated wafer herbivore dewormer score segment.

FIG. 4 depicts an exemplary dehydrated wafer herbivore dewormer scoring method.

FIG. 5 is a flowchart illustrating an exemplary manufacturing method of an exemplary dehydrated wafer herbivore dewormer.

FIG. 6 depicts an exemplary method of medical administration of an exemplary dehydrated wafer herbivore dewormer.

Like reference symbols in the various drawings indicate like elements.

### DETAILED DESCRIPTION OF ILLUSTRATIVE EMBODIMENTS

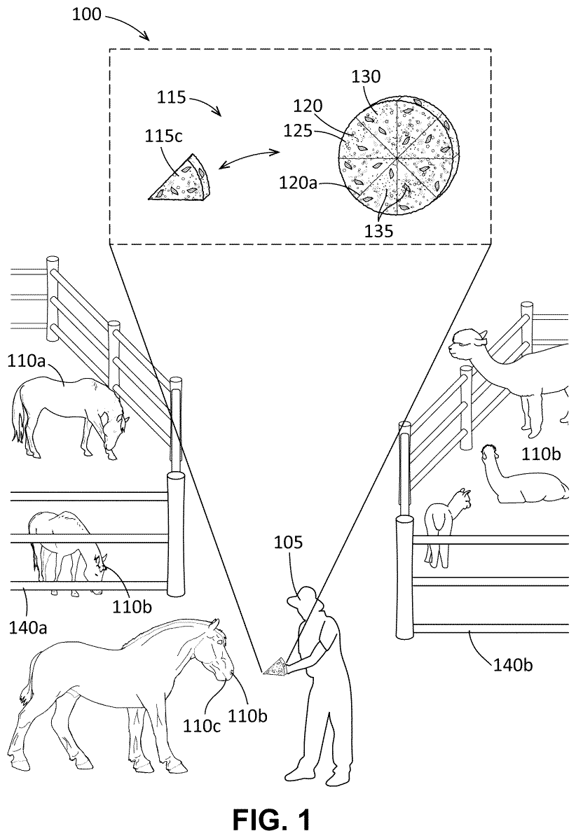

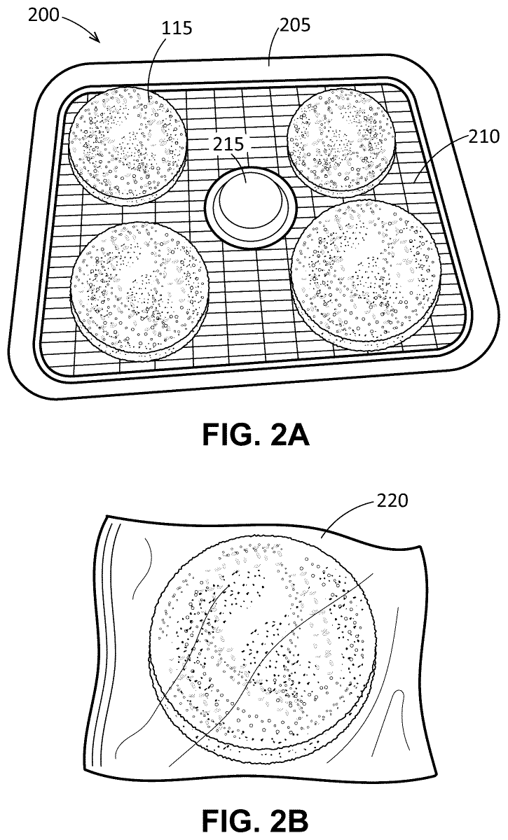

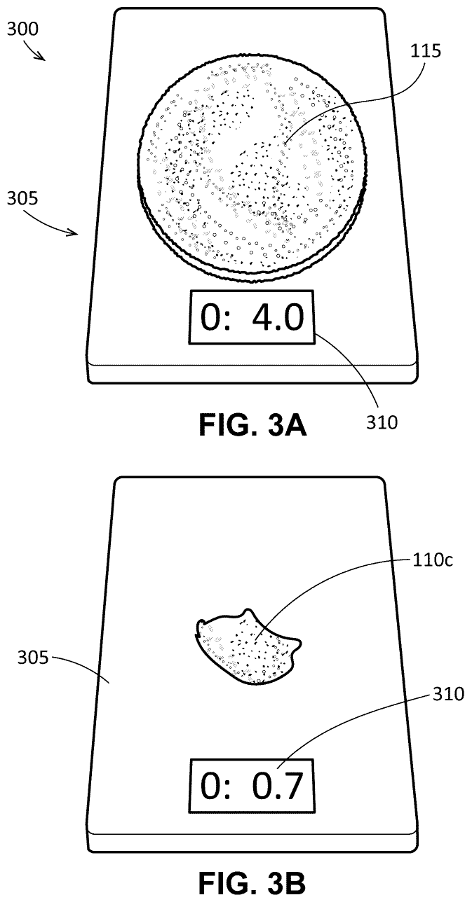

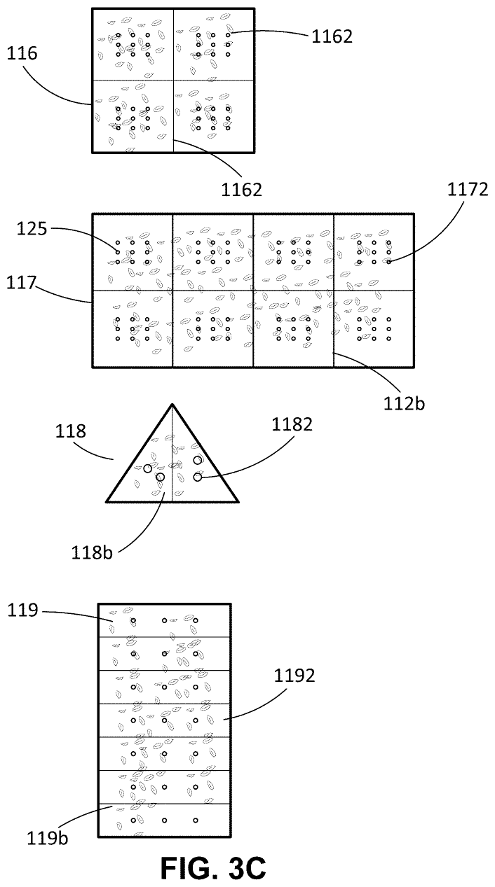

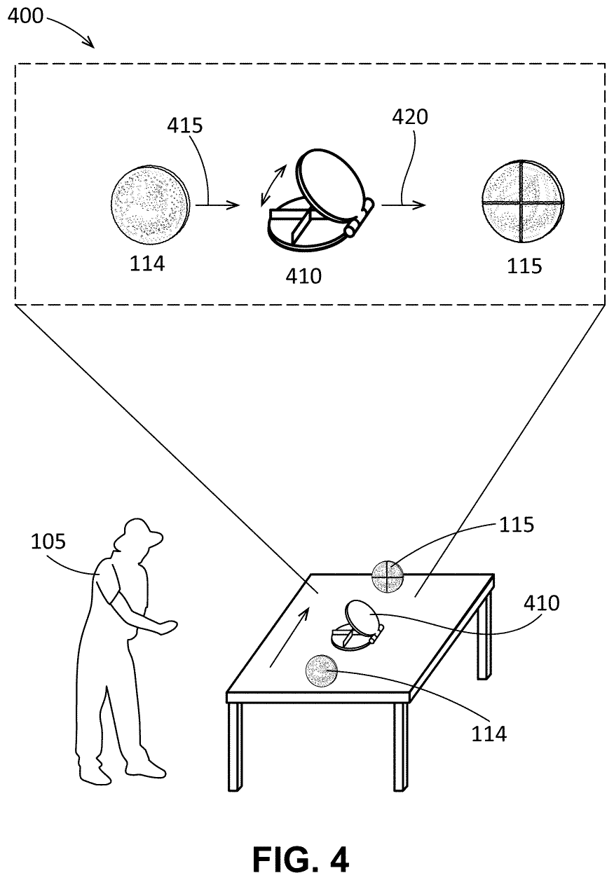

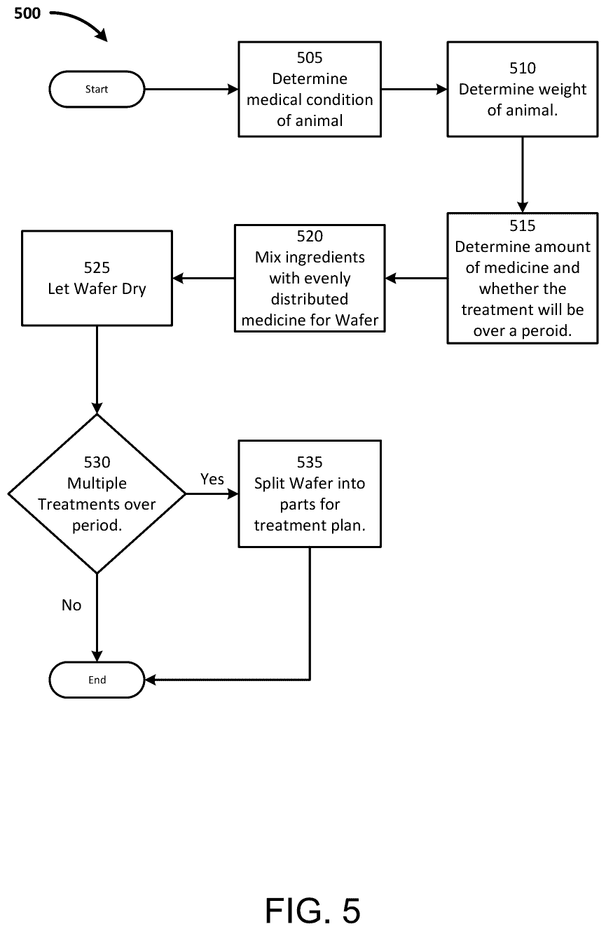

To aid understanding, this document is organized as follows. First, to help introduce a discussion of various embodiments, an exemplary dehydrated water herbivore dewormer (DWHD) is introduced concerning FIG. 1. Second, that introduction leads into a description concerning FIGS. 2A-2B the manufacture and storage of some exemplary embodiments of the DWHD. Third, concerning FIGS. 3A-3B, a method of weighing and score weighing is depicted. FIG. 3C depicts some exemplary embodiments of the DWHD and scoring of the DWHD. FIG. 4 depicts an exemplary method of manufacture of the DWHD. FIG. 5 depicts an exemplary application method of the DWHD. Finally, the document discusses further embodiments, exemplary applications, and aspects relating to applying a dehydrated equine wafer.

FIG. 1 depicts an exemplary dehydrated wafer herbivore dewormer (DWHD) employed in an illustrative use-case scenario 100. The illustrative use-case scenario 100 includes a user 105. The illustrative use-case scenario 100 includes an herbivore 110. The animal may, for example, include an equine. The animal may, for example, include a horse. The animal may, for example, include a donkey. The animal may, for example, include a mule. The animal may, for example, include an alpaca.

The illustrative use-case scenario 100 includes a depiction of an alpaca enclosure 140a. The alpaca enclosure includes alpacas 110b. The illustrative use-case scenario includes an horse enclosure 140b. The horse enclosure includes a small equine 110b. The horse enclosure 110c includes a larger equine 110c. A user may use a larger segment of the DWHD for the larger equine. A user may use a smaller segment of the DWHD for a smaller equine.

An herbivore owner may, for example, be able to purchase the DWHD for a single object herbivore dewormer for a designated species such as a horse. The herbivore owner may, for example, want each segment to have a predetermined dose for an animal (250 lbs.). The equine owner may, for example, want the DWHD to be edible for herbivores, such that the DWHD has a visible texture and/or a minimum ratio of roughage. The equine owner may, for example, want the DWHD to be scored to the DWHD can be subdivided into predetermined dosages.

The illustrative use-case scenario 100 includes a DWHD 115. The DWHD may, for example, include an edible animal anthelmintic wafer. The edible wafer contains palatable ingredients (e.g., barley, beet flour; medication (horse dewormer); binder; mold inhibitor and/or water). As an illustrative example, the DWHD may, for example, include palatable ingredients including barley mids and/or wheat flour. The palatable ingredients may, for example, include molasses. Palatable ingredients may, for example, be selected to be visually appealing to a purchaser. For example, palatable ingredients may include visible grains and/or other ingredients (e.g., selected according to expected appearance of palatable ingredients for a target animal to be treated).

In some implementations, the DWHD may, for example, include a binder (e.g., molasses, corn syrup). The binder may, for example, advantageously bind ingredients physically together. In some implementations, by way of example and not limitation, the binder may, for example, also act as a palatable ingredient.

The DWHD includes an appealing smell 115a. The smell may include an appealing smell that the herbivore recognizes. For example, for the animal 110 is a horse 110a. The horse may find the DWHD smell appealing due to the embodiment containing molasses aroma. The horse may find the DWHD smell appealing due the embodiment containing corn syrup aroma. The horse may find other aromas contained within the DWHD appealing.

The horse 110a may see the wafer with its sight 110b. The horse with its sight may see natural grains and roughage included into the DHWD. The horse may be appealed to eat the DWHD by the inclusion of the natural grains and roughage.

The horse 110a has a tense of taste and texture 110c. The horse may use its sense of taste and texture 110c to taste the DHWD taste 115b. The horse may use its tongue to taste a flavorant included in the DHWD. The horse may use its tongue to enjoy the texture of the DHWD. The horse may enjoy eating the DHWD, because it tastes great to the horse. The horse may not recognize the DHWD including an active ingredient. The active ingredient may include a dewormer medication blended into the DHWD. The horse may be medically treated based on receiving the medicine and appreciate the treatment due to it being included in a treat.

The DWHD 115 includes a segment 120. The DWHD 115 is segmented by segment lines 120a. Each wafer may, for example, be scored. For example, the wafer may be pressed in a mold. The mold may form score lines into the wafer. The score lines may radially divide a round wafer into predetermined sections (e.g., for ease of feeding).

The edible wafer 115 includes an active ingredient 125. As an illustrative example, an active ingredient 125 may, for example, include an anthelmintic (e.g., a dewormer such as fenbendazole or other parasiticides) distributed throughout the wafer. The edible wafer may include a single predetermined dose of anthelmintic. For example, the edible wafer may include a predetermined dosage for 250 pounds of animal (e.g., horse, equine). One or more edible wafers may advantageously be fed to an animal (e.g., a horse) to deworm the animal. The palatable ingredients may induce the animal (e.g., equine) to consume the edible wafer voluntarily. Accordingly, the animal may be easily treated for parasites without injection and without drenching. Each animal may be advantageously fed edible anthelmintic wafers treated with parasiticide with a known dosage instead of topdressing, in which each animal consumes a predetermined amount of anthelmintic. The animals may, for example, be in groups.

The active ingredient may include medication. The medication may, for example, include a horse dewormer. The medication may, for example, be in the form of a pellet. The pellets may, for example, include 0.5% (4,540 grams/ton) pellets of horse dewormer. The pellets may, for example, include 1.96% 17,740 (grams/tons) of horse dewormer. The wafer may, for example, include a flaked meal including horse dewormer. The medication may, for example, include fenbendazole as an active ingredient (e.g., Safeguard®, available through Merck Animal Health USA).

The DWHD 115 includes a segment 115c. The segment may be created for a single dose for a herbivore. The DWHD may be used for a treatment plan over an extended period time at predetermined administration times. Each segment may be give as a treatment for the predetermined administration times, so that after the DWHD has been consumed the treatment plan has been completed. Multiple DWHD may be used in an administration plan. One DWHD may for example provide enough treatment for one week. A herbivore may require 2 weeks of medication, so the two DWHD may be used.

In some embodiments, the wafer may be made by creating a dough of the ingredients, forming the wafer, and dehydrating the wafer. The dehydration process may occur at a lower temperature than pelletizing, which may advantageously maintain the full efficacy of the active anthelmintic ingredient.

The wafer may, for example, include a mold inhibitor to prevent mold growth in the wafer. The wafer may be packaged in predetermined dosage quantities (e.g., one wafer, two wafers in a package, or five wafers in a package).

In some implementations, a wafer may be formed from a predetermined ratio of ingredients. For example, palatable ingredients (e.g., roughage ingredients, starchy ingredients) may be at least 50% by weight of the wafer. The palatable ingredients may, for example, be at least 70% by weight of the wafer.

In some implementations, the binder may, for example, be at least 10% by weight of threshold wafer. For example, in some implementations, the binder may be at least 15% by weight. For example, the binder may be about 20% by weight, or more.

In some implementations, an active ingredient may be no more than 10% of the wafer by weight. In some implementations, for example, an active ingredient may be less than 5% of the wafer by weight. In some implementations, by way of example and not limitation, an active ingredient may be less than 3% of the wafer by weight.

In some implementations, the DWHD may, for example, include a flavorant (e.g., apple flavor). The flavor may be selected according to palatability tests to offset the taste of an active ingredient.

FIG. 2G are exemplary depictions 200 of the DWHD 115. The exemplary depictions 200 include racks 205. The DWHD may, for example, be dehydrated on racks 205. Multiple DWHDs may, for example, be dehydrated at the same time. The racks 205 include webs 210. The webs may allow for excess molasses, corn syrup, and/or other syrups added to the DWHD to strain through the webs of the racks. A topper 215 may be included in the racks 205. The topper 215 may facilitate additional stacking of racks such that the racks may be inserted into an oven. The topper may prevent the top of the DWHD from having contact with a bottom of a second rack above the DHWD while dehydrating.

The DWHD 115 may be stored in a sealable bag 220. The sealable bag may prevent the texture, taste, configuration that allows a horse to see the natural grains and roughage, and/or the smell of the DWHD. The sealable bag may allow the molasses and corn syrup not to become wet in rain, such that the dehydrated wafer herbivore dewormer does not become hydrated. A user may wish to hydrate the dewormer prior to giving it to the herbivore. A user may want to store the DWHD as dehydrated to increase the shelf life of the wafer.

Dehydration may, for example, be used to avoid high heat. High heat may, for example, interfere with the effectiveness of the medication. High heat may, for example, interfere with the quality of the product. The DWHD may, for example, be turned and mixed before forming into its shape. The DWHD may, for example, be stored in a plastic bag after dehydration. The DWHD may, for example, weigh 5 ounces. The DWHD may, for example, weigh 4 ounces. The DWHD weight may, for example, be 3 ounces. For example, a wafer of at least about 3 ounces may advantageously be subdivided while including sufficient volume to mix a predetermined ratio of active ingredient(s) to edible ingredients (e.g., for palatability).

In some implementations, by way of example and not limitation, a segment of a DWHD may, for example, weigh 0.7 ounces. A segment of a DWHD may, for example, weigh 1 ounce. A segment of a DWHD may, for example, weigh 0.5 ounces.

FIG. 3 depicts some exemplary shapes 300 and scores for a DWHD. For example, the exemplary shape 300 may include a multisegmented square DWHD 115a. For example, some exemplary shapes 300 may include a multisegmented rectangular DWHD 115b. Some exemplary shapes 300 may include a segmented triangle DWHD 115c. For example, some exemplary shapes 300 may include a segmented rectangular DWHD 115d.

In some implementations, a DWHD may, for example, be shaped according to a theme associated with a target animal. For example, for equines (e.g., horses, donkeys, ponies), in some embodiments, the DWHD may, for example, be shaped as a carrot. In some implementations, the DWHD may, for example, be shaped as an apple.

FIG. 3A-3B depicts a DWHD being weighed in an illustrative use case scenario 300. In the illustrative use case scenario 300, the DWHD 115 is being weighed. The illustrative use case scenario 300 includes a scale 305. The scale includes an output reading 310. A user may target a DWHD to weigh 4 ounces. A user may create a large batch of ingredients mixed together into a dough, create and weight the shape, and then dehydrate it. A user may then reweigh the DHWD after dehydrated. A user may wish the DWHD to weight a predetermined amount if the medication is evenly distributed throughout the dough. The user may estimate the amount of medicine in each DHWD based on an assumption of an evenly distributed active ingredient and/or medication in the dough prior to dehydrating. A score segment 115c may be weighed before given to the predetermined herbivore. A user may target each segment to weigh 0.7 ounces. A user may target a segment to weigh 0.75 ounces.

FIG. 3C depicts an some exemplary DWHD in an array 320. The array 320 includes a square DWHD 116. The square DHWD includes square segments 116a. An active ingredient 116b is stored within the segments 116a. The active ingredient may, for example, include a dewormer medication.

The array 320 includes a rectangular-squared segmented DWHD 117. The rectangular-square segmented DWHD 117 includes square segments 117a. An active ingredient 117 is stored within the segments 117a. The active ingredient may, for example, include a dewormer medication.

The array 320 includes a triangular DWHD 118. The triangular segmented DWHD 118 includes square segments 118a. An active ingredient 118b is stored within segments 118a. The active ingredient may, for example, include a dewormer medication.

The array 320 includes a rectangular-rectangular segmented DWHD 119. The rectangular-rectangular segmented DWHD 117 includes rectangular segments 119a. An active ingredient 119b is stored within the segments 119a. The active ingredient may, for example, include a dewormer medication.

The inclusion of the active ingredient within the wafer may advantageously allow for herbivore to consume the wafer by the allure of its senses, (e.g., sight, touch, smell, texture, taste), while the wafer includes dewormer medication such that the horse is able to receive medical treatment in a manner where the horses actively and positively engage in the receiving of the treatment. Some users treating their herbivores (e.g., horses) may, for example, regard their horse with a loving bond and may want to treat their horse in a manner in which the horse cooperates and associates a positive inclination with the user while receiving treatment. Some users may, for example, regard their horse with the same respect that they would give a family member, such as a sibling and/or child. The dehydrated edible wafer dewormer may, for example, improve a user's relationship with the equine, due to the equine regarding the dehydrated edible wafer dewormer as a treat. Some equines may, for example, regard dewormer medication by itself as unappetizing, and regard treatment without a wafer enclosing the medication as unpleasant.

In some embodiments, the palatable ingredients further include roughage ingredients and starchy ingredients. The palatable ingredients including roughage ingredients and starchy ingredients may, for example, appeal to herbivores such as horses and equines. In some embodiments, the active ingredient may include anthelmintic configured at a therapeutic does to treat or prevent internal parasitic infestations within herbivores, such as equines.

In some embodiments the edible wafer may, for example, include at least a 50% weight of palatable ingredients. The edible wafer in some embodiments may, for example, include at least 20% weight in binder. The binder may, for example, include yeast derived supplements, barley, molasses, corn syrup, water, and wheat flour. Mold inhibitors may, for example, be included in the binder such that the any mold associated with in the feed and/or pasture the herbivore consume is treated to prevent health issues, such as issue with the equines digestive system and/or gut. The edible wafer may, for example, include a flavoring. The flavoring may, for example, include an apple flavoring. The apple flavoring may advantageously appeal to the herbivore's senses of smell and/or taste to consume the edible wafer containing the active ingredient.

FIG. 4 depicts an exemplary use case 400 scenario incorporating a method of scoring a DWHD. The user 105 may segment the DHWD in the illustrative use-case scenario 400. The exemplary use case 400 includes an unsegmented DWHD 114. The unsegmented DWHD 114 may be positioned 415 to be segmented. The exemplary use case 400 includes a user 105. The exemplary use case 400 includes a press 410. The press may, for example, be operated by operating the press in a motion A that opens and closes the press. The process 420 may be used to segment the unsegmented DWHD 114. The press may, for example, include an automated machine to score the DWHD. The press may, for example, include a handheld device that operates as a press. The press may, for example, include stylized molds. The stylized molds may, for example, include a circular segmented mold, square segmented mold, rectangular segmented mold, triangular segmented mold, and/or carrot-shaped mold.

In some implementations, a press may, for example, include an interchangeable die. In some implementations, a press may include a hand press.

The exemplary use case 400 shows the process of the unsegmented circular DWHD 114 mold being pressed into a segmented DWHD 115. The DWHD may, for example, be round for packaging.

In some implementations, the DWHD 114 may, for example, be scored (e.g., as shown in FIG. 4. In some implementations, for example, the DWHD 114 may be pre-scored. For example, the DWHD 114 may be scored during a manufacturing process. For example, the scoring may be configured to correspond to specific dosing increments for the active ingredient(s).

FIG. 5 is a flowchart illustrating an exemplary method 500 of making a DWHD. In step 505, a user may, for example, determine the medical condition of the animal. The user may, for example, include a person, machine, software, controller, and/or artificial intelligence.

The medical condition may, for example, include the animal having parasites (e.g., nematodes). The medical condition may, for example, include the animal having heart disease. The medical condition may, for example, include a bacterial infection. In step 510, a user determines the amount of medicine required based on the weight of the animal. An entire wafer may, for example, be designed to treat a 250 lb. animal. To treat a larger animal, for example, a 1000 lb. animal 4 250 lb. wafer may, for example, be used. The wafers may, for example, allow a user to stage the administration of the medication.

In step 515, a user determines the amount of medicine required and whether the treatment will be over a period (i.e., administer ¼ a wafer of medication once a day for over a period of four days). In step 520, a user mixes the ingredients evenly with the medicine. In step 525, let the wafer dry and dehydrate. In step 530, if there are multiple treatments, a user splits the wafer into parts for the treatment plan in step 535. If there is no need for multiple treatments, wafer may, for example, be administered to the animal. A user may, for example, administer part of the wafer, depending on the dosage, may be administered to the animal.

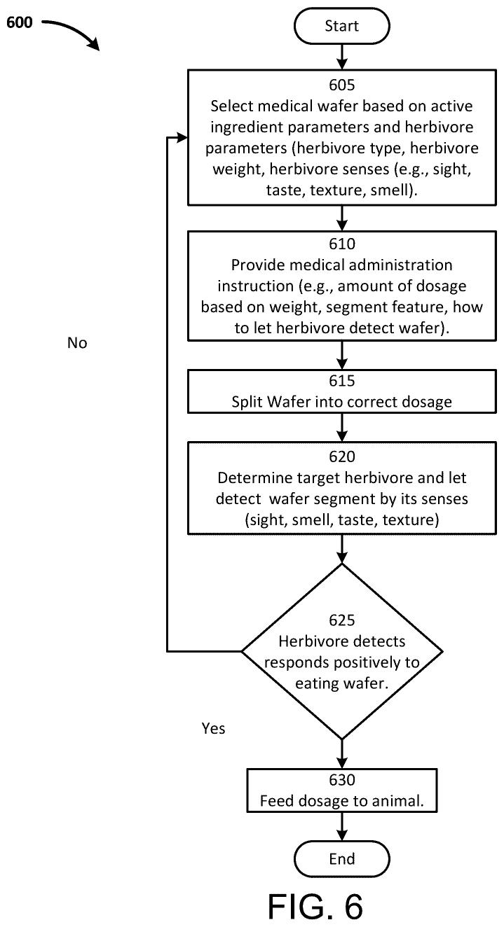

FIG. 6 is a flowchart illustrating an exemplary method 600 of application for a DWHD. In step 605, a user provides the medical wafer with the predetermined dosages. The user may select the medical wafer based on active ingredient parameters and the herbivore parameters. The parameters may, for example, include the herbivore type, the herbivore's weight, herbivore senses (e.g., sight, taste, texture, and/or smell). The wafer may, for example, be bought in packaging for a 250 lb. horse.

In step 610, a user provides medical administration instructions for the user. The medical administration instructions may, for example, include the amount of dosage based on weight, the segment features, the segments type, how to let the herbivore detect it (sight, taste, smell, etc.). The instructions may, for example, be included in the packaging. The instructions may, for example, be prescribed by an animal care provider. In step 615, a user splits the wafer into the correct dosage. For a 125 lb. juvenile horse (e.g., foal), the 250 lb. wafers may, for example, be split in half to feed the juvenile horse.

In step 620, determine the target herbivore (the herbivore requiring medical treatment) and let the target herbivore detect the wafer by its senses (sight, smell, taste, and/or texture. In step 625, if the herbivore responds positively to eating the wafer proceed to step 630. In step 630, a user feeds the dosage to the animal. If the herbivore is unable to sense and/or appreciate the waver, repeat with different composition of flavoring as required to receive a positive response.

In some embodiments, roughage may, for example, include high-fiber forage such as hay, grass, and haylage, is fundamental to a horse's diet, mirroring their natural grazing habits and ensuring optimal digestive health. This dietary component is not only essential for maintaining regular bowel movements and preventing constipation but also plays a crucial role in promoting a healthy microbial environment within the horse's large intestine. The fermentation of fiber in the hindgut produces volatile fatty acids, which serve as a significant energy source for the horse. Adequate roughage intake helps prevent digestive disturbances like colic and laminitis, supports weight management, and can improve overall well-being. By simulating natural foraging behavior, roughage consumption also contributes to mental health, reducing stress and boredom in stabled horses. Ensuring a consistent and plentiful supply of high-quality roughage is vital for sustaining the horse's health, providing a foundation for a balanced diet and supporting long-term vitality and performance.

In some embodiments, herbivores may, for example, sense the natural carbohydrates by their sense of sight, smell, taste, and/or texture. Some embodiments may advantageously be used to dispense the active ingredient for a dewormer medication. Some embodiments may include score lines to divide the active medication into predetermined amounts.

In some embodiments, palatable ingredients may, for example, be used in the edible wafer ensure the herbivores enjoy their food. The herbivores enjoyment of the edible wafer may, for example, be for optimal intake and digestion. For horses, highly favored ingredients may, for example, be used. Such palatable ingredients may, for example, include sweet feeds. Sweet feeds which are grain mixtures coated with molasses, as well as more nutrient-rich hays such as alfalfa and clover and treats like carrots and apples that provide a sweet and crunchy variety.

Alpacas may, for example, show a preference for high-quality grass hays like timothy or orchard grass. For example, alfalfa may be incorporated into the wafer in moderation due to its high protein and calcium content. Specialized pellets may, for example, be included in the wafer formulated for their unique dietary requirements. The essence of creating an appealing wafer for Alpacas may, for example, include offering a balanced variety of fresh, high-quality forages and supplements, tailored to their specific preferences and nutritional needs. The edible wafer may, for example, then promote healthy eating habits and supporting their overall well-being.

In some embodiments, incorporating natural carbohydrates such as molasses, flour, and binders like beet pulp and wheat flour into a horse's diet may be done with care to maintain health and energy levels without risking metabolic issues. Molasses may, for example, include a sweet by-product of sugar refining. Molasses may, for example, be used to enhance feed palatability and manage dust.

For context, in some embodiments, flour may, for example, be sourced from grains like oats, barley, or wheat. Flour incorporated in the edible wafer may, for example, provide a more concentrated energy source but should be balanced carefully with the horse's forage-based diet to avoid digestive disturbances.

Beet pulp and/or wheat flour may, for example, be used as a binder and fiber source. Beet pulp may, for example, offer soluble fiber that supports gut health and provides energy through fermentation in the hindgut, making it a safer option for energy supplementation. These carbohydrates may, for example, complement a forage-focused diet in the edible wafer, ensuring herbivores, such as horses, receive a well-rounded nutritional profile that supports their overall health, activity levels, and specific dietary requirements.

In some embodiments, wafers and mold inhibitors may, for example, serve roles in enhancing the quality and safety of horse feeds. Edible wafers may, for example, include blocks of feed ingredients. Edible wafers may, for example, provide a convenient and mess-free way to deliver nutrients, including forage, grains, and supplements. Edible Wafers may, for example, ensure horses receive a balanced diet even when fresh forage is scarce or when precise nutrient management is needed. Edible wafers may, for example, promote chewing and saliva production, supporting digestive health.

In some embodiments, edible wafers may, for example, include mold inhibitors. Mold inhibitors may, for example, be additives. Mold inhibitors may, for example, be used to preserve the freshness of horse feed and prevent the growth of harmful molds. Harmful molds may, for example, produce toxins detrimental to equine health. Mold inhibitors may, for example, be used to extend the shelf life of feeds and maintain their nutritional value, safeguarding horses against respiratory and digestive issues that can arise from consuming mold-contaminated feeds. Together, wafers and mold inhibitors may, for example, be used contribute to a practical, safe, and nutritious feeding strategy, ensuring horses receive the essential nutrients they need while minimizing risks associated with feed storage and spoilage.

Although various embodiments have been described concerning the figures, other embodiments are possible.

For example, in some implementations, a colorant may be provided. The colorant may, for example, correspond to a specific active ingredient. For example, a colorant may indicate to a user which active ingredient is in the DWHD. Such embodiments may, for example, advantageously assist a user in administering recommended and/or prescribed treatment regiments (e.g., anthelmintic rotation according to a veterinarian recommendation).

Although various embodiments are described with respect to anthelmintics, other active ingredients are contemplated. For example, some active ingredients may include antibiotics. In some implementations, active ingredients may include supplements (e.g., nutrients, probiotics).

In some implementations, for example, a mixture for a DWHD may be dynamically adapted to a target active ingredient. For example, a dosage of the active ingredient may be selected. Palatable ingredients and/or flavorants may be selected according to a target animal's known preferences and/or according to visual preferences of consumers purchasing for the target animal. A binder may be selected according to characteristics of the active ingredient and/or the palatable ingredient. The palatable ingredients, flavorant, and/or the binders ratio by weight of the DWHD may be selected at least partially according to palatability characteristics of the active ingredient. The ratio by weight of the binder may, for example, be adjusted to achieve a target consistency. A sample batch may be made to determine binding characteristics of the mixture when formed into a target shape (e.g., a wafer). The batch may be dehydrated. Analytics may be performed to verify ingredient and/or active ingredient concentration. An environmental chamber test may be performed for a predetermined period to test longevity (e.g., for molding). Mold inhibitor may be added, for example, until the environmental chamber test reaches a predetermined criterion (e.g., no mold under predetermined humidity, temperature, and/or time conditions). Ingredients may be adjusted to reach target consistency and/or analytic criteria. Ingredients may, for example, be further adjusted based on palatability trials.

Although an exemplary system has been described concerning FIG. 1-6, other implementations may be deployed in other industrial, scientific, medical, commercial, and/or residential applications. The DWHD may, for example, be used to treat show-animals. The DWHD may, for example, be used by animal care providers. The DWHD may, for example, be used by farmers to treat their animals. The DWHD may, for example, be easier to administer with less risk of injury for larger animals than administering a pellet without the DWHD. The DWHD may, for example, be easier to administer operationally than a shot. Multiple wafers may, for example, be used to treat an animal.

Although various embodiments have been described with reference to the figures, other embodiments are possible.

Although an exemplary system has been described with reference to FIG. 1-6, other implementations may be deployed in other industrial, scientific, medical, commercial, and/or residential applications. The DHWD may, for example, be used in trade shows for prize animals. The DHWD may, for example, be used at cattle shows. The DHWD may, for example, be used to treat animals during a rodeo. The DHWD may, for example, be used in educational training. The DHWD may, for example, be used commercially by a veterinarian professional.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A dehydrated equine dewormer wafer comprising:
an edible wafer composition comprising pieces of a first natural carbohydrate spread throughout the edible wafer configured such that the pieces of the first natural carbohydrate are visible on an exterior surface of the edible wafer;
a second natural carbohydrate coupled to at least some of the visible pieces of the first natural carbohydrate such that the natural carbohydrates may be sensed by an equine to be within the edible wafer composition; and,
an active ingredient evenly distributed throughout the edible wafer composition; and scorelines defining predetermined sections selectively separable from the edible composition wafer, configured such that each predetermined section comprises a predetermined amount of the active ingredient.

2. The equine dehydrated wafer dewormer of claim 1, wherein the second natural carbohydrate comprises a sweetener.

3. The equine dehydrated wafer dewormer of claim 2, wherein the sweetener comprises molasses.

4. The equine dehydrated wafer dewormer of claim 1, wherein the first natural carbohydrate comprises grain.

5. The equine dehydrated wafer dewormer of claim 1, wherein the first natural carbohydrate and the second natural carbohydrate physically bind together a base material, the active ingredient, and the pieces of the first natural carbohydrate.

6. The equine dehydrated wafer dewormer of claim 1, wherein the base material comprises at least 20% by weight of the edible wafer and comprises grain flour.

7. The equine dehydrated wafer dewormer of claim 1, wherein the active ingredient comprises at least 3% by weight of the edible wafer and comprises an anthelmintic comprising fenbendazole.

8. The equine dehydrated wafer dewormer of claim 1, wherein each section of the edible wafer comprises a dosage of active ingredient for a predetermined weight of equine.

9. The equine dehydrated wafer dewormer of claim 1, wherein the edible wafer further comprises a mold inhibitor spread throughout the wafer.

10. A dehydrated herbivore dewormer wafer comprising:
an edible wafer composition comprising pieces of a first natural carbohydrate spread throughout the edible wafer configured such that the pieces of the first natural carbohydrate are visible on an exterior surface of the edible wafer;
a second natural carbohydrate coupled to at least some of the visible pieces of the first natural carbohydrate such that the natural carbohydrates may be sensed by an herbivore to be within the edible wafer composition;
an active ingredient evenly distributed throughout the edible wafer composition; and scorelines defining predetermined sections selectively separable from the edible composition wafer, configured such that each predetermined section comprises a predetermined amount of the active ingredient.

11. The herbivore dehydrated wafer dewormer of claim 10, wherein the herbivore is an equine.

12. The equine dehydrated wafer dewormer of claim 1, wherein the second natural carbohydrate comprises a sweetener.

13. The equine dehydrated wafer dewormer of claim 2, wherein the sweetener comprises molasses.

14. The equine dehydrated wafer dewormer of claim 1, wherein the first natural carbohydrate comprises grain.

15. The equine dehydrated wafer dewormer of claim 1, wherein the first natural carbohydrate and the second natural carbohydrate physically binds together a base material, the active ingredient, and the pieces of the first natural carbohydrate.

16. The equine dehydrated wafer dewormer of claim 1, wherein the base material comprises at least 20% by weight of the edible wafer and comprises grain flour.

17. The equine dehydrated wafer dewormer of claim 1, wherein the active ingredient comprises at 3% by weight of the edible wafer and comprises an anthelmintic

18. The equine dehydrated wafer dewormer of claim 1, wherein the anthelmintic comprises fenbendazole.

19. The equine dehydrated wafer dewormer of claim 1, wherein each section of the edible wafer comprises a dosage of active ingredient for a predetermined weight of equine.

20. The equine dehydrated wafer dewormer of claim 1, wherein the edible wafer further comprises a mold inhibitor spread throughout the wafer.
